(19) United States
(12) Patent Application Publication
(10) Pub. No.: US 2024/0240253 A1
(43) Pub. Date: Jul. 18, 2024

(54) METHODS, KITS, AND SYSTEMS FOR MODULATING AND PREDICTING CHANGES IN P16, SENESCENCE, AND PHYSIOLOGICAL RESERVE

(71) Applicant: SAPERE BIO, INC., Research Triangle Park, NC (US)

(72) Inventor: Natalia MITIN, Durham, NC (US)

(21) Appl. No.: 18/289,605

(22) PCT Filed: May 5, 2022
(86) PCT No.: PCT/US2022/027824

§ 371 Date: Nov. 6, 2023

Related U.S. Application Data
(60) Provisional application No. 63/184,897, filed on May 6, 2021.

(51) Int. Cl. C12Q 1/6883; C12Q 1/6851

(52) U.S. Cl. CPC C12Q 1/6883; C12Q 1/6851; C12Q 2600/106; C12Q 2600/158

(57) ABSTRACT

The disclosure relates to methods, kits and systems for modulating and predicting changes in p16 and organismal cellular senescence.

### BACKGROUND

People age at different rates. Aging is a progressive process associated with the development of comorbidities and functional decline later in life. Being able to predict an individual's aging trajectory can aid in helping understand the effects of treatments and interventions on that individuals' aging trajectory and health status. Patient care often includes diagnostics to judge a patient's relative health and resilience, in an attempt to ascertain their ability to withstand insult and injury. Patients deemed more robust may be able to withstand more invasive or damaging interventions and may recover quickly without additional care. Patients deemed more vulnerable may require less invasive and damaging interventions, and may require additional palliative care for recovery. Thus, judging a patient's relative health is an important step in guiding that patient's medical decisions.

Traditional methods of assessing a patient's relative health often rely upon methods that include some combination of chronological age, existing comorbidities, and simple tests designed to measure cognition, endurance, strength, and physical ability.

Some more recent methods have leveraged molecular diagnostics, including, but not limited to, measuring levels of p16, to make better informed decisions regarding patient care, (see, e.g., Published U.S. Patent Application No. 20190032132 and U.S. Pat. No. 8,158,347). The application of those molecular diagnostics, while significantly better than relying on chronological age, is tailored to particular indications.

Thus, there remains a need for accurate diagnostic tests that help illuminate an individual's aging trajectory, and that can aid in predicting changes in markers of senescence, such as p16, which can impact an individual's health continuum and even change their aging trajectory.

Described herein are methods, compositions, systems, and kits that are useful for guiding patient choice when considering a broad set of medical interventions. The methods, compositions, systems, and kits disclosed herein are broadly useful for guiding decision making in a diverse set of unrelated medical interventions. Additionally, described herein, are methods, compositions, systems, and kits for decreasing cellular senescence and increasing the physiological reserve of certain identified subjects.

### SUMMARY

Other features and advantages of the concepts disclosed herein will be apparent from the following detailed description, the examples and the claims included herein.

In certain embodiments, a method of treating a subject to reduce the overall cellular senescent load of the subject is provided. In certain embodiments, a method of treating a subject to reduce the overall cellular senescent comprises: a) requesting a result of a clinical test, wherein the clinical test comprises obtaining a blood sample from the subject; b) detecting a level of gene expression of p16 in the sample for the subject and generating a value for p16; c) detecting a level of gene expression of CD28 in the sample for the subject and generating a value for CD28; d) detecting a level of gene expression of CD244 in the sample for the subject and generating a value for CD244; c) generating a composite score based on the value for p16, the value for CD28, and the value for CD244; wherein the composite score represents a prognostic value of the p16 levels in the subject in response to the intervention; and f) treating the subject with an intervention to reduce the overall cellular senescent load of the subject if the composite score indicates that the p16 levels of the subject may decrease as a result of the intervention.

In certain embodiments, a method of treating a subject to reduce the overall cellular senescent comprises generating a value for the interaction between CD28 and CD244, and combining it with the value for p16 to estimate the magnitude of change in p16 levels in the subject in response to the intervention. In certain embodiments, the generating a value for the interaction between CD28 and CD244 comprises: a) determining a population mean value of CD244 in a study population and subtracting the population mean value of CD244 in the study population from the CD244 expression level of the subject to generate a value representing the difference in CD244 expression between the subject and the population mean; b) determining a population mean value of CD28 in the study population and subtracting the population mean value of CD28 in the study population from the CD28 expression level of the subject to generate a value representing the difference in CD28 expression between the subject and the population mean; and c) multiplying the value representing the difference in CD244 expression between subject and the population mean, the value representing the difference in CD28 expression between subject and the population mean, and a parameter estimate of a CD28*CD244 interaction variable to generate a value for the interaction between CD28 and CD244.

In certain embodiments, a method of treating a subject to reduce the overall cellular senescent comprises isolating peripheral blood T lymphocytes from the blood sample.

In certain embodiments, a method of treating a subject to reduce the overall cellular senescent comprises generating a value for p16 comprises calculating a p16Age GAP Value for the subject. In certain embodiments, the generating a p16Age GAP Value comprises: (a) generating a p16 value for the subject from the level of gene expression of p16 in the sample; (b) converting the p16 value for the subject into a p16Age Value for the subject; and (c) generating a p16Age GAP Value for the subject by subtracting the chronological age of the subject from the p16Age Value of the subject.

In certain embodiments, a method of treating a subject to reduce the overall cellular senescent comprises administering one or more compounds that induce a senolytic effect. In certain embodiments, the one or more compounds comprises at least one of rapamycin, fisctin, metformin, canagliflozin, dapafliflozin, empagliflozin, ertugliflozin, roxadustat, molidustat, vadavustat, daprodustat, and dsatinib in combination with quercetin. In certain embodiments, a method of treating a subject to reduce the overall cellular senescent comprises one or more lifestyle interventions. In certain embodiments, the one or more lifestyle interventions comprises one or more of fasting, caloric restriction, dietary supplements, use of probiotics, an exercise regimen, and sleep monitoring.

In certain embodiments, a method of risk assessment for a subject considering the use of one or more interventions to lower the senescent load in the subject is provided. In certain embodiments, the method of risk assessment for a subject considering the use of one or more interventions to lower the senescent load in the subject comprises: a) requesting a result of a clinical test, wherein the clinical test comprises obtaining a blood sample from the subject; b) detecting a level of gene expression of p16 in the sample for the subject and generating a value for p16; c) generating a value for CD28 for the subject; d) generating a value for CD244 for the subject; c) generating a composite score based on the value for p16, the value for CD28, and the value for CD244; wherein the composite score represents a prognostic value of the p16 levels in the subject in response to the intervention; and f) comparing the composite score to a threshold value and determining that the subject should receive the intervention if the composite score falls on one side of the threshold value and determining that the subject should not receive the intervention if the composite score falls on the other side of the threshold value.

In certain embodiments, the method of risk assessment for a subject considering the use of one or more interventions to lower the senescent load in the subject comprises generating a value for the interaction between CD28 and CD244, and combining it with the value for p16 to estimate the magnitude of change in p16 levels in the subject in response to the intervention. In certain embodiments, the generating a value for the interaction between CD28 and CD244 comprises: a) determining a population mean value of CD244 in a study population and subtracting the population mean value of CD244 in the study population from the CD244 expression level of the subject to generate a value representing the difference in CD244 expression between the subject and the population mean; b) determining a population mean value of CD28 in the study population and subtracting the population mean value of CD28 in the study population from the CD28 expression level of the subject to generate a value representing the difference in CD28 expression between the subject and the population mean; and c) multiplying the value representing the difference in CD244 expression between subject and the population mean, the value representing the difference in CD28 expression between subject and the population mean, and a parameter estimate of a CD28*CD244 interaction variable to generate a value for the interaction between CD28 and CD244.

In certain embodiments, the method of risk assessment for a subject considering the use of one or more interventions to lower the senescent load in the subject comprises isolating peripheral blood T lymphocytes from the blood sample.

In certain embodiments, the method of risk assessment for a subject considering the use of one or more interventions to lower the senescent load in the subject comprises generating a score for p16 comprises calculating a p16Age GAP Value for the subject. In certain embodiments, the generating a p16Age GAP Value comprises: (a) generating a p16 value for the subject from the level of gene expression of p16 in the sample; (b) converting the p16 value for the subject into a p16Age Value for the patient; and (c) generating a p16Age GAP Value for the subject by subtracting the chronological age of the subject from the p16Age Value of the subject.

In certain embodiments, the method of risk assessment for a subject considering the use of one or more interventions to lower the senescent load in the subject comprises administering one or compounds that induce a senolytic effect. In certain embodiments, the one or more compounds comprises at least one of rapamycin, fisetin, metformin, canagliflozin, dapafliflozin, empagliflozin, ertugliflozin, roxadustat, molidustat, vadavustat, daprodustat, and dsatinib in combination with quercetin. In certain embodiments, the method of risk assessment for a subject considering the use of one or more interventions to lower the senescent load in the subject comprises one or more lifestyle interventions. In certain embodiments, the one or more lifestyle interventions comprises one or more of fasting, caloric restriction, dietary supplements, use of probiotics, an exercise regimen, and sleep monitoring.

In certain embodiments, the method of risk assessment for a subject considering the use of one or more interventions to lower the senescent load in the subject comprises a threshold value that is set such that the subject receives the intervention if p16 levels are projected to decrease and does not receive the intervention if p16 levels are projected to increase or remain the same.

### BRIEF DESCRIPTION OF THE DRAWINGS

FIG. 1 shows changes in p16 expression (log 2) after intervention (v2) as compared to prior to intervention (v1), as described in Example 1. Distribution of changes in p16 in each patient and the summary statistics for the entire cohort are shown.

FIG. 2 shows that changes in p16 expression (log 2) after intervention correlate with p16 expression prior to intervention, as described in Example 1.

FIG. 3 shows a model fit of p16 levels prior to intervention used to predict changes in p16, as described in Example 1. Actual values for p16 changes vs predicted by the model are plotted. The line and shaded areas represent the mean and 95% confidence interval, respectively.

FIG. 4 shows a model fit of interactions between CD28 and CD244 gene expression levels prior to intervention used to predict changes in p16, as described in Example 1. Actual values for p16 changes versus changes predicted by the CD28*CD244 model are plotted. The line and shaded areas represent the mean and 95% confidence interval, respectively.

FIG. 5 shows a model fit of p16 expression levels prior to intervention and interactions between CD28 and CD244 gene expression levels prior to intervention to predict changes in p16, as described in Example 1. Actual values for p16 changes versus changes predicted by the model are plotted. The line and shaded areas represent the mean and 95% confidence interval, respectively.

FIG. 6 shows a model fit of p16Age GAP measured prior to intervention and interactions between CD28 and CD244 gene expression levels prior to intervention to predict changes in p16, as described in Example 1. Actual values for p16 changes vs changes predicted by the CD28*CD244 model are plotted. The line and shaded areas represent the mean and 95% confidence interval, respectively.

FIG. 7 shows receiver operating characteristic (“ROC”) analysis of the algorithm developed and described in Example 1 with respect to the binary endpoint of decrease in p16 when measured after intervention as compared to before the intervention. Change in p16 expression equal or less than −0.4 was considered a decrease.

FIG. 8 shows ROC analysis of the algorithm developed and described in Example 1 with respect to the binary endpoint of increase in p16 when measured after intervention as compared to before the intervention. Change in p16 expression equal or higher than 0.4 was considered an increase.

FIG. 9 shows a model fit of p16 expression levels prior to intervention (pre) and interactions between CD28 and CD244 gene expression levels prior to intervention to predict changes in p16 (post-pre), as described in Example 2. Actual values for p16 changes vs changes predicted by the model are plotted. The line and shaded areas represent the mean and 95% confidence interval, respectively.

### DETAILED DESCRIPTION

Section headings used herein are for organizational purposes only and are not to be construed as limiting the subject matter described. Unless otherwise defined, all technical and scientific terms used herein have the same meaning as commonly understood by one of ordinary skill in the art to which this disclosure belongs. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Unless defined otherwise, technical and scientific terms used herein have the same meaning as commonly understood by one of ordinary skill in the art to which the concepts described herein belong. The following terms, unless otherwise indicated, shall be understood to have the following meanings:

As used herein, a “subject” can be an individual that is a human or other animal. A “patient” refers to a class of subjects who is under the care of a treating physician (e.g., a medical doctor or veterinarian). The subject can be male or female of any age. Exemplary and non-limiting subjects include, humans, rabbits, mice, rats, horses, dogs, and cats. In one embodiment, the subject has undergone or will undergo a surgical intervention, such as a cardiovascular surgical intervention described herein. In other embodiments, the subject has been treated or will be treated with a chemotherapeutic, for example, paclitaxel.

The term “sample,” as used herein, refers to a composition that is obtained or derived from a subject. The sample can be whole blood or a blood sample that has been fractionated. The sample may be peripheral blood leukocytes including neutrophils, cosinophils, basophils, lymphocytes, and monocytes. In some embodiments, the sample is a peripheral blood lymphocyte selected from B cells, T cells and NK cells. In some embodiments, the sample is a peripheral blood T lymphocyte (e.g., a T cell) or a subset of T cells (e.g., CD3+, CD8+ cells). In some embodiments, the sample is a tissue biopsy. In certain embodiments, the sample comprises genetic information. In certain embodiments, the sample comprises at least one of proteins, metabolites, steroids, hormones, sugars, salts, or other physiological components.

As used herein, the term “gene” refers to a nucleic acid that encodes an RNA, for example, nucleic acid sequences including, but not limited to, structural genes encoding a polypeptide. The term “gene” also refers broadly to any segment of DNA associated with a biological function. As such, the term “gene” encompasses sequences including but not limited to a coding sequence, a promoter region, a transcriptional regulatory sequence, a non-expressed DNA segment that is a specific recognition sequence for regulatory proteins, a non-expressed DNA segment that contributes to gene expression, a DNA segment designed to have desired parameters, or combinations thereof. A gene can be obtained by a variety of methods, including cloning from a biological sample, synthesis based on known or predicted sequence information, and recombinant derivation from one or more existing sequences.

The term “gene expression” generally refers to the cellular processes by which a biologically active polypeptide is produced from a DNA sequence and exhibits a biological activity in a cell. As such, gene expression involves the processes of transcription and translation, but also involves post-transcriptional and post-translational processes that can influence a biological activity of a gene or gene product. These processes include, but are not limited to RNA synthesis, processing, and transport, as well as polypeptide synthesis, transport, and post-translational modification of polypeptides. Additionally, processes that affect protein-protein interactions within the cell can also affect gene expression as defined herein. In some embodiments, the phrase “gene expression” refers to a subset of these processes. As such, “gene expression” refers in some embodiments to transcription of a gene in a cell type or tissue. Thus, the phrase “expression level” can refer to a steady state level of an RNA molecule in a cell, the RNA molecule being a transcription product of a gene. Expression levels can be expressed in whatever terms are convenient, and include, but are not limited to absolute and relative measures. For example, an expression level can be expressed as the number of molecules of mRNA transcripts per cell or per microgram of total RNA isolated from cell. Alternatively, or in addition, an expression level in a first cell can be stated as a relative amount versus a second cell (e.g., a fold enhancement or fold reduction), wherein the first cell and the second cell are the same cell type from different subjects, different cell types in the same subject, or the same cell type in the same subject but assayed at different times (e.g., before and after a given treatment, at different chronological time points, etc.).

The term “gene product” generally refers to the product of a transcribed gene, such as a protein, peptide, or enzyme. The term “gene product” may also refer to non-proteins, such as a functional RNA (fRNA), for example, micro RNAs (miRNA), piRNAs, ribosomal RNAs (rRNAs), transfer RNAs (tRNAs), and the like.

The terms “template nucleic acid” and “target nucleic acid” as used herein each refers to nucleic acids isolated from a biological sample as described herein above.

The term “target-specific primer” refers to a primer that hybridizes selectively and predictably to a target sequence, for example and not limitation, a target sequence present in an mRNA transcript derived from the p16INK4a/ARF locus A target-specific primer can be selected or synthesized to be complementary to known nucleotide sequences of target nucleic acids.

The term “primer” as used herein refers to a contiguous sequence comprising in some embodiments about 6 or more nucleotides, in some embodiments about 10-20 nucleotides (e.g. a 15-mer), and in some embodiments about 20-30 nucleotides (e.g. a 22-mer). Primers used to perform the method of the presently disclosed subject matter encompass oligonucleotides of sufficient length and appropriate sequence so as to provide initiation of polymerization on a nucleic acid molecule.

The term “sensitivity” refers to a measurement of the proportion of actual positively identified results in a binary test (e.g., the proportion of individuals identified as having a condition who are correctly identified as having the condition in a diagnostic test).

The term “specificity” refers to a measurement of the proportion of actual negatively identified results in a binary test (e.g., the proportion of individuals identified as not having a condition that are correctly identified as not having the condition in a diagnostic test).

The term “negative predictive value” refers to the proportion of identified negative results that are actually negative for a condition in a diagnostic test.

The term “positive predictive value” refers to the proportion of identified positive results that are actually positive for a condition in a diagnostic test.

The term “threshold” refers to a specific level at which a measured parameter has been established. The exact threshold values and the diagnostic correlations to a particular state vary depending on the gene expression measuring assay and can be determined empirically by comparison to reference samples that have been shown to be positive and negative for acquiring the particular state. Expression levels above this threshold and below this threshold are indicative of a positive or negative diagnostic outcome, respectively. A specific cutoff for the threshold may be set depending on the desired sensitivity and specificity for a subject population.

The terms “predicting” and “likelihood” as used herein does not mean that the outcome is occurring with 100% certainty. Instead, it is intended to mean that the outcome is more likely occurring than not. Acts taken to “predict” or “make a prediction” can include the determination of the likelihood that an outcome is more likely occurring than not.

The terms “composite score” or “composite result” refer to a score that is generated through analyzing two or more variables. In certain embodiments, variables represent individual scores, and in certain embodiments, represent scores from individual biomarkers. Examples of variables used to calculate a composite score include, but are not limited to, measurements of gene expression, measurements of chronological age, measurements of protein levels, measurements of organ and systems function such as cognition, or ability to walk as ascertained by physical or written testing, genotyping, other measurements of health or senescence based on testing, measurements of molecules in bodily fluids, such as urine or blood, measurements of molecules in the lungs, such as oxygen levels, and measurements of other biomarkers. In certain embodiments, a variable is a measure of immunosenescence in an organism. In certain embodiments, a variable is a measure of cellular senescence. In certain embodiments, a variable is a measure of chronic disease of one or more specific organs or systems in an organism diagnosed by standard clinical testing. In certain embodiments, a variable is a measure of the function of one or more specific organs or systems in an organism. In certain embodiments, a variable is a measure of the overall function of an organism and is not organ or system specific. In certain embodiments, two or more variables are used to calculate a first composite score, which is itself a variable that is then combined with other variables to calculate a second composite score. In certain embodiments, a threshold is established using a composite score. In certain embodiments, a composite score is generated for a subject. In certain such embodiments, the composite score generated for a subject is compared to the threshold established for that composite score.

In certain embodiments, a composite score is generated using one or more algorithms. In certain embodiments, algorithms for generating a composite score can include variables that are given identical or different weights, depending on how the algorithm is constructed. For example, and not limitation, a variable that represents a certain biomarker might be given a weight equivalent to 50% of the score even if there are three other different variables used to generate the composite score. In certain other embodiments with the same four biomarkers, each biomarker might be given an equivalent weight (25%) when generating a composite score. In certain embodiments, variables can be added together to create a composite score. In certain such embodiments, variables can have either a positive or negative value when used to calculate the composite score. For example, and not limitation, a composite score might be calculated by adding together the weighted variables A and B, and then subtracting the weighted variable C. In certain embodiments, interactions between variables can be calculated. For example, and not limitation, individual variables can be calculated from a scored value for a subject minus a population average for that variable, and two or more such variables can be multiplied together.

In certain embodiments, a variable can be excluded from a composite score if the value associated with that variable falls outside of a given range. For example, and not limitation, a variable may only be part of a composite score if it falls between 0.3 and 0.7 units. If that variable exceeds 0.7 units or is less than 0.3 units, it is excluded from the composite score. In certain embodiments, the value of a variable can function as a gateway to one or more different algorithms. For example, and not limitation, if a subject is homozygous wild-type or heterozygous at a given locus, a composite score is calculated using algorithm A. If a subject is homozygous mutant at that locus, a composite score is calculated using algorithm B. In certain embodiments, gateway variables can be used that result in three or more arms, for example, and not limitation, if a variable is scored between 0 and 0.3 units, a composite score is calculated using algorithm A, if a variable is scored greater than 0.3 but less than 0.9 units, a composite score is calculated using algorithm B, if a variable is scored at or above 0.9 units, a composite score is calculated using algorithm C. In certain embodiments, a gateway variable can also function as a way to exclude a subject. For example, and not limitation, if a subject is homozygous wild-type or heterozygous at a given locus, a composite score is calculated using algorithm A. If that subject is homozygous mutant at that locus, no composite score is calculated.

In certain embodiments, algorithms for generating a composite score can include statistical methods for determining values. For example, and not limitation, algorithms can include ordinary least squares regression analysis, Deming regression analysis, non-linear regression modeling, partition analysis, neural network analysis, decision tree analysis, probability theory methods, and other methods known to those of skill in the art.

In certain embodiments, an algorithm includes parameter estimates. Parameter estimates are values that are calculated to determine the relative contribution of a variable in relationship to other variables to predict a pre-defined outcome in regression analysis. In certain embodiments, parameter estimates are calculated from a cohort and function as coefficients in the algorithm to provide different weights to the variables in the algorithm. In certain embodiments, a parameter estimate is calculated for the CD28*CD244 interaction variable. In certain embodiments, a parameter estimate is calculated for the p16 variable. In certain embodiments, a parameter estimate is calculated for the p16 Age Gap variable.

The term p16 refers to the gene encoded by the cyclin dependent kinase inhibitor 2a (CDKN2A) transcript variant 1. This gene corresponds to the National Center for Biotechnology Information (NCBI) accession numbers NM_000077.4 (mRNA) and NP_000068.1 (protein). As used herein, p16^{INK4a} refers also to p16 or any other common gene synonym.

The terms “p16Age” and “p16Age Value” refer to a value assigned to a subject based on that subject's p16 levels relative to the p16 values of a given cohort of subjects. In certain embodiments, p16Age is based on a statistical analysis of an individual's p16 levels relative to the cohort's p16 levels (see, e.g., International Application No. PCT/US2021/062747). In certain embodiments, p16Age is calculated by converting log 2p16 expression values into the units of age using linear regression formula. In certain embodiments, p16Age for a subject may differ from the subject's chronological age. For example, and not limitation, the p16Age of a subject may be 85, while that subject's chronological age may only be 45. In such a case, the subject's p16Age would exceed the subject's chronological age by 40 years. In certain embodiments, p16Age in a subject is the same, or at least approximately the same, as the chronological age of the subject. In certain embodiments, p16Age for a subject can be greater than or lesser than the chronological age for that subject. In certain embodiments, p16Age is a variable that is useful for predicting the onset of a disease or a condition. In certain embodiments, p16 Age is a variable that is useful for predicting changes to p16 in response to a treatment or intervention.

Because linear regression analysis is used to derive p16Age, it can at times greatly exceed the reasonable limits of a subject's lifespan. Thus, in certain embodiments, a subject's p16Age may have a value well over 100 years of age. In certain embodiments, one can use alternative computational models (See, e.g., Tsygankov et al., Proc. Natl. Acad. Sci. (2009)) that demonstrate p16 change with age to calculate p16Age to reflect that a given subject's lifespan is not infinite and p16 values saturate with age.

The terms “p16Age GAP” and “p16Age Gap Value” refer to the difference between a subject's p16Age Value and the chronological age of the subject. To derive the differential between p16 and chronological age, one converts log 2p16 expression values into age using linear regression calculations from a scatter plot of log 2p16 vs chronological age. The slope is derived from linear regression analysis using the least square method. The intercept is determined as the age at which the p16 value is zero. The resulting value of p16 converted to calendar year units is then used to calculate p16Age GAP by subtracting chronological age. In certain embodiments, p16Age GAP for an individual can be a positive value. In certain embodiments, p16Age GAP for an individual can be a negative value. In certain embodiments, p16Age GAP for an individual can be zero. In certain embodiments, p16Age GAP is a variable that is useful for predicting the onset of a vulnerability to an adverse event or disease. In certain embodiments, p16 Age GAP is a variable that is useful for predicting changes to p16 in response to a treatment or intervention.

The term “physiological reserve” refers to the ability of an individual, a physiological system, or an organ to withstand or recover from insult or injury. While physiological reserve declines with age, a variety of other factors can cause a decline in the reserve. In certain embodiments, health varies significantly between individuals of the same chronological age based on the different physiological reserve of the different individuals. In some cases, physiological reserve differs between individuals of similar chronological age based on each individual's genetics. In some cases, physiological reserve differs between individuals of similar chronological age but different life experiences. Life experiences that can affect physiological reserve include, but are not limited to, consumption of alcohol, smoking, stress, chronic inflammation, environmental exposure, radiation, chemotherapy, exposure to poisons, and dietary decisions. In certain embodiments, markers of cellular senescence can be used to help determine physiological reserve.

In certain embodiments, physiological reserve can be measured using markers of cellular senescence. The term “senescence” refers to the process or condition of deterioration over time. The term “cellular senescence” refers to a cell losing the ability to divide. In many cases, cellular senescence represents a permanent cell cycle arrest in which cells remain metabolically active and adopt characteristic phenotypic changes. The onset of cellular senescence can occur in response to stress stimuli, such as, for example, cell stress caused by inflammation. Markers of cellular senescence include, but are not limited to, p14^{ARF}, p16^{INK4a}, Klotho, p15^{NK4b}, MDM2, p21, p53, macroH2A, IL-6, IGFBP-2, PAI-1, HMGB1, p38 MAPK, SA-β-Gal, markers of DNA methylation, and telomere length.

When discussing cellular senescence in the art, there are two different concepts to distinguish. Sometimes when the art discusses cellular senescence, it is referring to the cellular senescent load, or the quantity of cells in an organism or tissue that are senescent. While the cellular senescent load increases with age, the rate of an increase can be changed by preferentially removing the senescent cells, such as through apoptosis or the use of senolytics. Individual senescent cells expressing p16, however, are permanently arrested, and that arrest is not reversible. But p16 expression levels of the organism or tissue as a whole can be decreased by preferentially removing these cells. Thus, where those skilled in the art refer to reducing cellular senescence, they are really referring to reducing the cellular senescent load in the organism or tissue.

In certain embodiments, cellular senescence, as measured by p16 levels, is an indicator of physiological reserve. For example, and not limitation, in the case of p16, expression of p16 is not detected in young cells, increases exponentially with chronological age (doubling approximately every 8 years in humans), and is potently activated by age-promoting stimuli, including, but not limited to, cigarette smoking, physical inactivity, radiation, cytotoxic chemotherapy administration, chronic HIV infection, and bone marrow transplantation. Exposure to these toxic stimuli can cause acceleration of aging phenotypes and can be monitored through expression of p16 in various tissues, including T cells in peripheral blood. In certain embodiments, measuring p16 levels in peripheral blood, and from T cells in particular, provides an overall view of organismal aging (Sec, e.g. U.S. Pat. No. 8,158,347). In contrast, measuring p16 from a specific tissue, such as an organ, may provide insight into the health of that organ, but not necessarily the overall health of the organism. In addition, p16 levels can increase in some organs in response to insult or injury. Thus, in certain embodiments, measuring p16 levels in peripheral blood provides a more comprehensive measure of organismal senescence state or physiological reserve than measuring p16 from one or more individual tissues.

Prior to the work described herein, some experts considered cellular senescence to be irreversible without intervention with pharmacological agents described to have senolytic properties (see, e.g., Song et al., Cells (2020)). Even in the case of senolytics, a reduction in cellular senescence is achieved by removing senolytic cells and reducing the overall cellular senescence load rather than by reversing the cellular senescence in individual cells, causing them to regain the ability to divide.

Senescent cells are resistant to apoptosis and accumulate in tissues. Recent evidence suggests that senescent cells can be cleared by the immune system. Therefore, accumulation and turnover of senescent cells exists in a balance. As the organism ages (or receives age-accelerating stimuli), the rate of accumulation of senescent cells can increase or the immune system ability to clear senescent cells declines (immunosenescence). In addition, interaction between senescent and immune cells affects immune system function. Senescent cells recruit and make immune cells senescent and dysfunctional via Senescent-Associated Secretory Phenotype (often referred to as ‘SASP’). As a result of all the above scenarios, senescent cells accumulate at a higher rate, causing decline in physiological reserve and aging.

In certain embodiments, the turnover of senescent cells by the immune system is due to the reactivation of the apoptosis program. In certain embodiments, senolytics induce turnover of senescent cells by inducing them to undergo apoptosis and these apoptotic cells are usually cleared by the immune system.

In certain embodiments, caloric restriction and/or diet restriction can induce cell stress that activates nutrient-sensing pathways and activates molecular processes that can produce a senolytic effect. In certain embodiments, potential mechanisms of action include, but are not limited to, enhancement of the immune system to improve targeting and turnover of senescent cells; turnover of the senescent cells themselves by allowing apoptosis or clearance by Natural Killer (NK) cells; blocking SASP secretion from existing senescent cells and thus preventing formation of new senescent cells by paracrine stimulation.

The senescence program is driven by a complex interplay of signaling pathways. To promote and support cell cycle arrest, p16 and the p53 (TP53) target p21 (CDKN1A), inhibits cyclin-dependent kinases (CDKs), thereby preventing phosphorylation of the retinoblastoma protein (pRb) and thus in turn suppressing the expression of proliferation-associated genes (see, e.g., Narita et al, 2003, 2006; Collado et al, 2007). In addition, the nuclear factor kappa B protein complex (NF-KB) acts as a master regulator of SASP expression and therefore affects both the microenvironment of senescent cells and their immune surveillance (see, e.g., Acosta et al, 2008; Krizhanovsky et al, 2008; Xue et al, 2011; Lasorella et al, 2014). Clearance of senescent cells by the immune system helps limit their prolonged retention in tissues, a trait that might derive from their intrinsic resistance to apoptosis (see, e.g., Yosef et al, 2016). The anti-apoptotic BCL-2 family members BCL-W, BCL-XL, and BCL-2 were shown to facilitate the resistance of senescent cells to apoptosis (see, e.g., Chang et al, 2016; Yosef et al, 2016). However, the contribution of pathways that regulate the formation of senescent cells to the resistance of these cells to cell death has yet to be determined. On one hand, senescent cells cannot accumulate p53 protein to the levels required for apoptosis (Seluanov et al, 2001). On the other hand, the p53 target p21, via its ability to promote cell cycle inhibition, can protect some cells from apoptosis (Abbas & Dutta, 2009).

The term “immunosenescence” refers to the gradual deterioration of the immune system due to increasing age and exposure to insults. In certain embodiments, immunosenescence renders the immune system slow to respond to stimuli (although it is still capable of being activated), increasing susceptibility to both infections and age-related diseases. In certain embodiments, immunosenescence can be reversible. In contrast, the cellular senescence state of individual cells, for example and not limitation, as measured by p16 levels in T cells, is not reversible. Thus, in certain embodiments, increased expression of p16 in T cells can indicate cellular senescence, but not necessarily indicate immunosenescence. Immunosenescence, also a factor in aging, is characterized by changes in T cell subsets (decrease in naïve T cells, increase in memory T cells), lack of T cell activation (CD28 negative), and changes in expression of certain genes that suggest T cell exhaustion, for example and not limitation, CD8, CD4, CD28, CD57, CD140, CD244, CD160, and LAG3. While T cells can simultaneously display features of cellular senescence and immunosenescence, these processes correlate only weakly. Thus, in certain embodiments, cellular senescence and immunosenescence represent distinct processes that both contribute to aging and inflammatory phenotypes across tissue types. In certain such embodiments, measuring biomarkers of both immunosenescence and cellular senescence and combining those measurements into a composite score provides more information than measuring cellular senescence and immunosenescence separately. In certain embodiments, because cellular senescence load (the quantity of senescent cells) is regulated by the immune system, combining measurements of cellular senescence and immune health/immunosenescence provides a more complete picture of the overall senescence state and physiological reserve of a subject.

CD28 is expressed on the surface of T cells and CD28 signaling is involved in the initial activation of naïve CD8+ and CD4+ T cells. CD28 in humans is expressed on approximately 80% of CD4+ T cells and 50% of CD8+ T cells. And the loss of CD28 expression from both CD8+ cells and CD4+ cells has been associated with immunosenescence and physical frailty (Ng. et al., 2015).

CD244 is a transmembrane cell surface receptor expressed on NK cells and some T cells. In humans, CD244 is alternatively spliced resulting in two different receptors that differ in their extracellular domains. CD244 signaling is complex and involves both activating and inactivating effects (See, e.g., Agresta et al., Frontiers in Immunology (2018)). CD48 is a known ligand for CD244.

T cell cellular senescence, which can be measured by measuring p16 levels, can be distinguished from T cell anergy and T cell exhaustion that occurs as immunosenescence progresses. T cell anergy is a hyporesponsive state in T cells which is triggered by excessive activation of the T cell receptor (TCR) and either strong co-inhibitory molecule signaling or limited presence of concomitant co-stimulation through CD28. In certain embodiments, T cell anergy can be measured by measuring CD28 expression levels. T cell exhaustion occurs after repeated activation of T cells during chronic infection.

In certain embodiments, T cell exhaustion manifests with several characteristic features, such as progressive and hierarchical loss of effector functions, sustained upregulation and co-expression of multiple inhibitory receptors, altered expression and use of key transcription factors, metabolic derangements, and a failure to transition to quiescence and acquire antigen-independent memory T cell homeostatic responsiveness. Although T cell exhaustion was first described in chronic viral infection in mice, it has also been observed in humans during infections such as HIV and hepatitis C virus (HCV), as well as in cancer. While T cell exhaustion prevents optimal control of infections and tumors, modulating pathways overexpressed in exhaustion—for example, and not limitation, by targeting programmed cell death protein 1 (PD1) and cytotoxic T lymphocyte antigen 4 (CTLA4)—can reverse this dysfunctional state and reinvigorate immune responses.

T cell signaling is complex and involves many different factors and genes that work in parallel, contradictory, synergistic, or competing signaling pathways. Accordingly, in certain embodiments, a measurement of gene expression of a single gene may not be very informative as a marker for measuring immunosenescence. In certain embodiments, a measurement of immunosenescence is performed by measuring gene expression from two or more genes involved in immunosenescence and comparing the relative levels of those genes to produce a composite score that better represents the immunosenescence state of the subject than measuring any of those same genes separately. For example and not limitation, CD244 signaling is complex and is only partially understood and probably has effects on multiple different cellular processes, but by comparing CD244 expression levels with expression levels of other markers of immunosenescence, a composite score can be generated that better represents the immunosenescence state of a subject than measuring CD244 alone. As another example and not limitation, CD28 expression is associated with T-cell anergy and CD244 expression is associated with T-cell exhaustion, therefore by looking at both CD28 expression and CD244 expression, one can capture different processes that are involved in immunosenescence and gain more insight into the immunosenescence state of a subject than could be achieved by measuring a single marker. In certain embodiments, generating a score by measuring both CD28 and CD244 provides a composite score that represents immunosenescence and, optionally, that composite score can be combined with other markers of cellular senescence to create a second composite score that can be used to guide treatment of a subject.

Both immunosenescence and cellular senescence involve the complex interplay of multiple signal transduction pathways, and can be thought of as progressive processes. For example, and not limitation, the immunosenescence of an organism's immune system can become more or less senescent over time, depending on which signal transduction pathways are activated and how those signal transduction pathways interact with other active and inactive signal transduction pathways that affect immunosenescence.

In certain embodiments, understanding cellular senescence progression and/or immunosenescence progression comprises evaluating multiple different markers in a composite score. Similarly, composite scores can be used to evaluate the likelihood that a particular subject will respond negatively or positively to a proposed treatment or intervention. In certain embodiments, at least one marker in a composite score evaluates the general health of the individual, such as, for example, one or more markers for physiological reserve or senescence. In certain embodiments, at least one marker in a composite score comprises evaluating one or more specific markers specific to one or more particular organs or tissues. For example, and not limitation, when considering risk of developing a kidney related disease, one can include a marker for kidney function. In certain embodiments, a method of generating a composite score comprises generating a composite score from both markers of general health and markers for specific tissues and/or organs.

In certain embodiments, a p16Age GAP is calculated for a patient. In certain embodiments, a p16Age GAP is calculated by subtracting the chronological age of a patient from a p16Age Value determined for that patient. In certain embodiments, the p16Age GAP can be used to guide intervention or treatment decisions for a patient.

In certain embodiments, composite scores are generated comprising variables for cellular senescence and variables for immunosenescence. For example and not limitation, in certain embodiments, composite scores are generated comprising variables for p16Age GAP, CD28, and CD244. In certain such embodiments, those composite scores can guide treatment decisions, including whether a subject should be given a senolytic and whether a subject should receive a treatment that is likely to increase senescence based on levels of markers of cellular senescence. In certain embodiments, a composite score may reveal that an intervention may increase a subject's p16 levels, and that intervention can be avoided.

In certain embodiments, an individuals' treatment can be personally tailored based on the likelihood that their p16 levels will increase, stay the same, or decrease. In certain such embodiments, subjects that can benefit from caloric restriction are identified and separately treated from those that will not see such a benefit. In certain such embodiments, subjects that are likely to see an increase in senescent markers from caloric restriction are identified and treated to avoid that increase. Methods of caloric restriction include cutting calories below what a subject typically consumes over a given time period. In certain embodiments, caloric restriction includes cutting calories by 5% or more, 10% or more, 15% or more 20% or more, 25% or more, 30% or more, 35% or more, 40% or more, 45% or more, 50% or more, 55% or more, 60% or more, 65% or more, 70% or more, 75% or more, or 80% or more. In certain embodiments, methods of caloric restriction include regiments of intermittent fasting. Examples of intermittent fasting include, but are not limited to, intermittent fasting with periods of feeding and fasting in each day (for example, and not limitation, 16 hours of feeding and 8 hours of fasting); restricting feeding to one meal a day; fasting on clear liquids only (e.g. water) 1-3 days with some periodicity; and diets aimed at stabilizing blood glucose (low carb keto, Mediterranean or other plant-based) consumed at libitum, and combinations thereof.

In certain embodiments, the methods, biomarkers, algorithms, and techniques described herein can be used to screen different interventions to discover interventions that are effective at decreasing cellular senescence, immunosenescence, or both cellular senescence and immunosenescence. For example, and not limitation, one can use the methods, biomarkers, algorithms, and techniques described herein to screen senolytic therapies or lifestyle interventions for efficacy in decreasing cellular senescence, immunosenescence, or both cellular senescence and immunosenescence. In certain embodiments, one can use the methods, biomarkers, algorithms, and techniques described herein to compare the effectiveness of potential interventions against each other. For example, and not limitation, one can compare the effectiveness of various dietary interventions or the effectiveness of various senolytics, or combinations of both senolytics and dietary interventions. Examples of molecules with senolytic effect that can be used in combination with the methods discussed herein include, but are not limited to, rapamycin and its analogs, fisetin, dasatinib in combination with quercetin (“D+Q”), metformin, SGLT2 inhibitors, including, but not limited to, canagliflozin, dapafliflozin, empagliflozin, and ertugliflozin, and HIF inhibitors, including, but no limited to, roxadustat, molidustat, vadavustat, and daprodustat. In certain embodiments, the methods, biomarkers, algorithms, and techniques described herein can be used to guide lifestyle interventions for subjects. Examples of lifestyle interventions include, fasting, caloric restriction, dietary supplements, use of probiotics and other dietary interventions, exercise, and sleep monitoring.

In certain embodiments, the methods, biomarkers, algorithms, and techniques described herein can be used to distinguish subjects that may benefit from an intervention from those subjects that may not benefit from that intervention. For example, and not limitation, a subject with higher levels of p16 may potentially benefit from an intervention that will lower those levels of p16 following the intervention, whereas a subject with already low levels of p16 will likely not see any benefit from the intervention, or may even see p16 levels rise due to the stress associated with the intervention or risk of adverse events associated with those interventions. Thus, the methods, biomarkers, algorithms, and techniques described herein can be used to balance the risk of intervention versus the potential benefit of the intervention, allowing one to better guide a subject's treatment.

In certain embodiments, a decision to administer a senolytic treatment can be guided by considering the probability that treatment will promote a desired senolytic effect, an undesired effect of p16 levels rising, or a scenario where the probability of change in either direction is low. In certain embodiments, if a subject's p16 levels are predicted to increase from baseline by a value higher than the analytical precision of measurement of p16 expression, the treatment would be counter indicated. In certain embodiments, if a subject's p16 levels are predicted to decrease from baseline by a value higher than the analytical precision of measurement of p16 expression, the treatment would be considered beneficial. In certain embodiments, if the predicted change in a subject's p16 levels falls within the precision of measurement, there is a 95% probability that treatment will not have a senolytic effect and treatment should not be used for those purposes.

In certain embodiments, subject selection can be used to guide study design for identifying senolytics and interventions that have a senolytic effect by selecting for patients with high p16 (which have the opportunity of seeing a significant senolytic effect) and excluding patients with low p16 where any senolytic effect would likely be insignificant, negligible, or counterproductive. In certain embodiments, by excluding subjects that are unlikely to receive a senolytic effect because their p16 levels are already low, one can enrich in subjects that are candidates for senolytic effects which can help facilitate identification of effective senoltyics and interventions with a senolytic effect.

As shown herein, cellular senescence can be reversed in certain individuals with certain treatments, including, but not limited to caloric restriction and the administration of senolytics. In certain such embodiments, the reversal of cellular senescence is achieved by reducing the overall cellular senescent load (the quantity of senescent cells in the individual) through mechanisms such as, for example and not limitation, apoptosis and targeting of senescent cells by NK or CD8+ cells. In certain embodiments, individuals are identified as candidates for benefiting from caloric restriction by measuring markers of at least one of cellular senescence and immunosenescence. In certain embodiments, individuals are identified as candidates for benefiting from caloric restriction or the administration of senolytics by measuring p16, CD28, and CD244. Other embodiments described herein comprise identifying individuals that will not benefit from caloric restriction or the administration of senolytics.

The methods described herein can be used to detect gene expression in a biological sample, and more particularly in a blood sample in a subject (e.g., a human patient). Genc expression levels can be determined in whole blood samples or, more typically, the whole blood sample can be manipulated or fractionated prior to determining gene expression level. Manipulation of blood samples is well known in the art and can include separation of red blood cells from white blood cells and plasma, or separation of various cell types from each other, including isolating specific white blood cells, or more specifically isolating T-lymphocytes, and measuring gene expression levels in the isolated cell type(s). In some embodiments, gene expression levels of p16^{INK4a} are measured from a sample of isolated T-lymphocytes.

The level of gene expression can be determined using a variety of molecular biology techniques that are well known in the art. For example, if the expression level is to be determined by analyzing RNA isolated from the biological sample, techniques for determining the RNA expression level include, but are not limited to, Northern blotting, nuclease protection assays, quantitative PCR (e.g., digital RT-PCR and/or real time quantitative RT-PCR), branched DNA assay, direct sequencing of RNA by RNA seq, nCounter gene expression technology (NanoString Technologies), single cell sequencing, reserve transcription loop-mediated isothermal amplification (RT-LAMP), and droplet digital PCR technology. In some embodiments, expression levels are determined by real time quantitative PCR (RT-PCR) employing specific PCR primers for the p16^{INK4a} gene. PCR primers for p16^{INK4a} are described, for example, in U.S. Pat. No. 8,158,347, and such description is incorporated herein by reference.

Alternatively, expression levels can be determined by analyzing protein levels in a biological sample using antibodies. Methods for quantifying specific proteins in biological samples are known in the art. Representative antibody-based techniques include, but are not limited to, immunodetection methods such as ELISA, Western blotting, in-cell Western, bead-based immunoaffinity, immunoaffinity columns, and 2-D gel separation. In certain embodiments, at least one of immunosenescence and cellular senescence can be measured by mRNA expression or by counting cells by using techniques such as, for example and not limitation, flow cytometry and single cell analysis.

Methods for nucleic acid isolation can comprise simultaneous isolation of total nucleic acid, or separate and/or sequential isolation of individual nucleic acid types (e.g., genomic DNA, cell-free RNA, organelle DNA, total cellular RNA, mRNA, polyA+RNA, rRNA, tRNA) followed by optional combination of multiple nucleic acid types into a single sample. Such isolation techniques are known to those skilled in the art. Nucleic acids that are to be used for subsequent amplification and labeling can be analytically pure as determined by spectrophotometric measurements or by analysis following electrophoretic resolution (BioAnalyzer, Agilent). The nucleic acid sample can be free of contaminants such as polysaccharides, proteins, and inhibitors of enzyme reactions. When an RNA sample is intended for use as probe, it can be free of nuclease contamination. Contaminants and inhibitors can be removed or substantially reduced using resins for DNA extraction (e.g., CHELEX™ 100 from BioRad Laboratories, Hercules, Calif., United States of America) or by standard phenol extraction and ethanol precipitation. Isolated nucleic acids can optionally be fragmented by restriction enzyme digestion or shearing prior to amplification.

Various methods for designing primers for specific nucleic acid sequences of interest are well known in the art. Primers for amplifying p14^{ARF} and p16^{INK4a} separately can be designed based upon the specific sequences chosen. For example, p14^{ARF} and p16^{INK4a} transcripts have a unique exon 1 but share exon 2. Therefore, to design primers specific for p14^{ARF} or p16^{INK4a}, a forward primer can be selected for each unique exon 1 and a reverse primer can be selected for the common exon 2. Conversely, suitable primers may be designed to amplify the shared portion of exon 2 of p14^{ARF} and p16^{INK4a} to determine the expression level of both genes together. In addition, it can be beneficial to design primers that flank the exon/intron junction, for example, to eliminate amplification signal from genomic DNA contamination in RT-PCR reaction. Non-limiting exemplary primers for detecting p14^{ARF} and p16^{INK4a} are described in U.S. patent application Ser. No. 16/078,476.

In some embodiments of the present disclosure, the abundance of specific mRNA species present in a biological sample (for example, mRNA extracted from peripheral blood T lymphocytes) is assessed by quantitative RT-PCR. Standard molecular biological techniques are used in conjunction with specific PCR primers to quantitatively amplify those mRNA molecules corresponding to the gene or genes of interest. Methods for designing specific PCR primers and for performing quantitative amplification of nucleic acids including mRNA are well known in the art. See e.g., Heid et al., 1996; Sambrook & Russell, 2001; Joyce, 2002; Vandesompele et al., 2002. In some embodiments, a technique for determining expression level includes the use of the TAQMAN® Real-time Quantitative PCR System (ThermoFisher Scientific, United States of America).

Specific primers for genes of interest (e.g., p16) are employed for determining expression levels of these genes. In some embodiments, the expression level of one or more housekeeping genes (e.g., YWHAZ) are also determined in order to normalize a determined expression level. In one aspect, the level of expression of p16 from a sample may be normalized to a housekeeping gene from a batch of combined samples. In another aspect, the level of expression of p16 from a sample may be normalized to a housekeeping gene from the same sample.

The primers and probes used for amplification and detection may include a detectable label, such as a radiolabel, fluorescent label, or enzymatic label. See, U.S. Pat. No. 5,869,717, hereby incorporated by reference. In certain embodiments, the probe is fluorescently labeled. Fluorescently labeled nucleotides may be produced by various techniques, such as those described in Kambara et al., Bio/Technol., 6:816-21, (1988); Smith et al., Nucl. Acid Res., 13:2399-2412. (1985); and Smith et al., Nature, 321: 674-679, (1986), the contents of each of which are herein incorporated by reference herein for their teachings thereof. The fluorescent dye may be linked to the deoxyribose by a linker arm that is easily cleaved by chemical or enzymatic means. There are numerous linkers and methods for attaching labels to nucleotides, as shown in Oligonucleotides and Analogues: A Practical Approach, IRL Press, Oxford, (1991); Zuckerman et al., Polynucleotides Res., 15: 5305-5321, (1987); Sharma et al., Polynucleotides Res., 19:3019, (1991); Giusti et al., PCR Methods and Applications, 2:223-227, (1993); Fung et al. (U.S. Pat. No. 4,757,141); Stabinsky (U.S. Pat. No. 4,739,044); Agrawal et al., Tetrahedron Letters, 31:1543-1546, (1990); Sproat et al., Polynucleotides Res., 15:4837, (1987); and Nelson et al., Polynucleotides Res., 17:7187-7194, (1989), the contents of each of which are herein incorporated by reference herein for their teachings thereof. Extensive guidance exists in the literature for derivatizing fluorophore and quencher molecules for covalent attachment via common reactive groups that may be added to a nucleotide. Many linking moieties and methods for attaching fluorophore moieties to nucleotides also exist, as described in Oligonucleotides and Analogues, supra; Guisti et al., supra; Agrawal et al., supra; and Sproat et al., supra.

The products of the Quantitative PCR employed in the TAQMAN® Real-time Quantitative PCR System can be detected using a probe oligonucleotide that specifically hybridizes to the PCR product. Typically, this probe oligonucleotide is labeled at the 5′ and/or 3′ ends with one or more detectable labels described herein. In some embodiments, the 5′ end is labeled with a fluorescent label and the 3′ end is labeled with a fluorescence quencher. In some embodiments, the 5′ end is labeled with tetrachloro-6-carboxyfluorescein (TET™; Applera Corp., Norwalk, Conn., United States of America) and/or 6-FAM™ (Applera Corp.) and the 3′ end includes a tetramethylrhodamine (TAMRA™; Applera Corp.), NFQ. BHQ, and/or MGB quencher.

Additional exemplary and non-limiting detectable labels may be attached to the primer or probe and may be directly or indirectly detectable. The exact label may be selected based, at least in part, on the particular type of detection method used. Exemplary detection methods include radioactive detection, optical absorbance detection, e.g., UV-visible absorbance detection, optical emission detection, e.g., fluorescence; phosphorescence or chemiluminescence; Raman scattering. Preferred labels include optically-detectable labels, such as fluorescent labels. Examples of fluorescent labels include, but are not limited to, 4-acetamido-4′-isothiocyanatostilbene-2,2′disulfonic acid; acridine and derivatives: acridine, acridine isothiocyanate; 5-(2′-aminoethyl)aminonaphthalene-1-sulfonic acid (EDANS); 4-amino-N-[3-vinylsulfonyl)phenyllnaphthalimide-3,5 disulfonate; N-(4-anilino-1-naphthyl)maleimide; anthranilamide; BODIPY; alexa; fluorescein; conjugated multi-dyes; Brilliant Yellow; coumarin and derivatives; coumarin, 7-amino-4-methylcoumarin (AMC, Coumarin 120), 7-amino-4-trifluoromethylcouluarin (Coumaran 151); cyanine dyes; cyanosine; 4′,6-diaminidino-2-phenylindole (DAPI); 5′5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansylchloride); 4-dimethylaminophenylazophenyl-4′-isothiocyanate (DABITC); eosin and derivatives; cosin, cosin isothiocyanate, erythrosin and derivatives; erythrosin B, erythrosin, isothiocyanate; cthidium; fluorescein and derivatives; 5-carboxyfluorescein (FAM), 5-(4,6-dichlorotriazin-2-yl)aminofluorescein (DTAF), 2′,7′-dimethoxy-4′5′-dichloro-6-carboxyfluorescein, fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′tetramethyl-6-carboxyrhodamine (TAMRA); tetramethyl rhodamine; tetramethyl rhodamine isothiocyanate (TRITC); riboflavin; rosolic acid; terbium chelate derivatives; Atto dyes, Cy3; Cy5; Cy5.5; Cy7; IRD 700; IRD 800; La Jolta Blue; phthalo cyanine; and naphthalo cyanine. Labels other than fluorescent labels are contemplated by the methods described herein, including other optically-detectable labels.

Other methodologies for determining gene expression levels can also be employed, including but not limited to Amplified Antisense RNA (aaRNA) and Global RNA Amplification (Van Gelder et al., 1990; Wang et al., 2000; U.S. Pat. No. 6,066,457 to Hampson et al.). In accordance with the methods of the presently disclosed subject matter, any one of the above-mentioned PCR techniques or related techniques can be employed to perform the step of amplifying the nucleic acid sample and/or quantitating the expression of a particular target nucleic acid. In addition, such methods can be optimized for amplification of a particular subset of nucleic acid (e.g., specific mRNA molecules versus total mRNA), and representative optimization criteria and related guidance can be found in the art. See Williams, 1989; Linz et al., 1990; Cha & Thilly, 1993; McPherson et al., 1995; Roux, 1995; Robertson & Walsh-Weller, 1998.

For any particular biomarker, graphical distributions of gene expression levels for subjects are not completely distinct but instead will overlap. Therefore, any diagnostic test that measures a biomarker does not absolutely distinguish low-risk patients from patients that are at high-risk for adverse cellular senescence progression or adverse immunosenescence progression with 100% accuracy. The graphical area of overlap correlates to a range of gene expression levels wherein the test cannot distinguish low-risk or normal from high risk. Thus, the developer of the test must select a threshold level of expression from the area of overlap and conclude that levels above the threshold are considered at risk and expression levels below the threshold are considered to be normal or not at risk. The smaller the area of overlap, the more accurate the diagnostic test will be.

Determining the exact threshold value to determine those at risk and those not at risk will depend upon the assay format being developed. In certain embodiments, threshold values may be determined empirically using techniques well known by those skilled in the art. For example, and not limitation, a threshold for determining a risk of increased cellular senescence or increased p16 expression levels may be determined by obtaining a suitable biological sample from a population of patients in which one or more biomarkers may be measured prior to intervention or treatment.

One exemplary and non-limiting way to determine the ability of a particular test to distinguish two populations can be by using receiver operating characteristic (ROC) analysis. To draw a ROC curve, the true positive rate (TPR) and false positive rate (FPR) are determined as the decision threshold is varied continuously. Since TPR is directly correlated with sensitivity and FPR is inversely correlated with specificity (1-specificity), the ROC graph is sometimes called the sensitivity vs (1-specificity) plot. The area under the ROC curve is a measure of the probability that the perceived measurement will allow correct identification of a condition. A perfect test will have an area under the ROC curve of 1.0 whereas a random test will have an area of 0.5. Therefore, any actual diagnostic test analyzed using ROC analysis will have an area under the ROC curve somewhere between 0.5 and 1.0. The closer to 1.0 the curve is, the more accurate the test is.

ROC analysis is often used to select a threshold that provides an acceptable level of specificity and sensitivity to distinguish a subpopulation with a particular condition or state from a subpopulation without that particular condition or state. In general, optimal threshold is the point on the ROC curve closest to the upper left corner (100% sensitivity; 100% specificity). However, depending on the particular condition or state or patient population a more detailed description of ROC analysis and its use for evaluating diagnostic tests and predictive models can be found in the art, for example, in Zou et al., Circulation. 2007; 115:654-657.

In addition to the measurement of area under the curve (AUC), the effectiveness of a given biomarker to predict or identify a particular condition or state can be estimated through several additional measures of diagnostic test accuracy (described in Fischer et al., Intensive Care Med. 29: 1043-51, 2003). These measures include sensitivity and specificity, likelihood ratios (LR), and diagnostic odds ratios (OR).

In certain embodiments, the specificity of the assay for identifying risk ranges from about 30% to about 100%, including each integer within the specified range. In certain embodiments, the specificity of the assay for identifying risk ranges from about 50% to about 100%, including each integer within the specified range. In certain embodiments, the specificity of the assay for identifying risk ranges from about 70% to about 100%, including each integer within the specified range. In certain embodiments, the specificity of the assay for identifying risk ranges from about 30% to about 50%, including each integer within the specified range. In certain embodiments, the specificity of the assay for identifying risk ranges from about 40% to about 60%, including each integer within the specified range. In certain embodiments, the specificity of the assay for identifying risk from about 50% to about 70%, including each integer within the specified range. In certain embodiments, the specificity of the assay for identifying risk from about 60% to about 80%, including each integer within the specified range. In certain embodiments, the specificity of the assay for ranges from about 70% to about 90%, including each integer within the specified range. In certain embodiments, the specificity of the assay is about 30%, about 35%, about 40%, about 45%, about 50%, about 55%, about 60%, about 65%, about 70%, about 75%, about 80%, about 85%, about 90%, about 95%, or even about 100%.

In certain embodiments, the sensitivity of the assay for identifying risk ranges from about 30% to about 100%, including each integer within the specified range. In certain embodiments, the sensitivity of the assay for identifying risk ranges from about 50% to about 100%, including each integer within the specified range. In certain embodiments, the sensitivity of the assay for identifying risk ranges from about 70% to about 100%, including each integer within the specified range. In certain embodiments, the sensitivity of the assay for identifying risk ranges from about 30% to about 50%, including each integer within the specified range. In certain embodiments, the sensitivity of the assay for identifying risk ranges from about 40% to about 60%, including each integer within the specified range. In certain embodiments, the sensitivity of the assay ranges from about 50% to about 70%, including each integer within the specified range. In certain embodiments, the sensitivity of the assay for identifying risk ranges from about 60% to about 80%, including each integer within the specified range. In certain embodiments, the sensitivity of the assay for identifying risk ranges from about 70% to about 90%, including each integer within the specified range. In certain embodiments, the sensitivity of the assay is about 30%, about 35%, about 40%, about 45%, about 50%, about 55%, about 60%, about 65%, about 70%, about 75%, about 80%, about 85%, about 90%, about 95%, or even about 100%.

In certain embodiments, the ROC curve area is an area ranging from about 0.5 to about 1, including each fractional integer within the specified range. In one aspect, the ROC curve area is greater than at least 0.5, at least 0.6, at least 0.7, at least 0.8, at least 0.9, or even at least 0.95.

In certain embodiments, the suitable positive likelihood ratio is a ratio (calculated as sensitivity/(1-specificity)) of at least 1, at least 2, at least 3, at least 5, at least 10; and a negative likelihood ratio (calculated as (1-sensitivity)/specificity) of less than 1, less than or equal to 0.5, less than or equal to 0.3, less than or equal to 0.1; an odds ratio different from 1, at least about 2 or more, at least about 3 or more, at least about 4 or more, at least about 5 or more, or even at least about 10 or more.

In certain embodiments, markers that predict or identify a particular condition or state can be coupled with other markers to generate a composite score. Methods for combining assay results can comprise, but are not limited to, the use of multivariate logistic regression, n-of-m analysis, decision tree analysis, calculating hazard ratios, and other methods known to those skilled in the art. In certain embodiments, a composite result which is determined by combining individual markers measured prior to intervention, can be treated as if it itself is a marker; that is, a threshold determined for a composite result as described herein for individual markers, and the composite result can be used in to calculate odds ratio for individual patients.

In another embodiment, biomarkers can be used to stratify a subject population and identify a population where measurements of p16Age GAP combined with measurements of other biomarkers are used as components of a composite scare to predict or identify a particular condition or state with the most sensitivity, specificity, and positive likelihood. Exemplary markers can be markers of organ function, cellular senescence status, immunosenescence status, inflammation status, or can be genetic markers.

Some embodiments described herein are non-naturally occurring DNA sequences that are useful in predicting or identifying a particular condition or state in a subject. In certain embodiments, these non-naturally occurring DNA sequences contain at least one sequence segment that crosses at least one exon-exon boundary or untranslated region-exon boundary without containing the intervening intronic sequences. Therefore, these DNA sequences do not naturally occur. As would be understood by a person of ordinary skill, these non-naturally occurring DNA sequences may be generated from a naturally occurring biological sample, such as RNA through reverse transcriptase-PCR followed by amplification with a suitable primer. In some aspects, the non-naturally occurring DNA sequence further comprises a non-natural or modified DNA base known by those skilled in the art.

The non-naturally occurring DNA sequences described herein may comprise between 10 and 1,000 bases, including each integer within the specified range. In one aspect, the non-naturally occurring DNA sequence comprises between 10 and 500 bases, including each integer within the specified range. In another aspect, the non-naturally occurring DNA sequence comprises between 10 and 300 bases, including each integer within the specified range. In another aspect, the non-naturally occurring DNA sequence comprises between 10 and 200 bases, including each integer within the specified range. In another aspect, the non-naturally occurring DNA sequence comprises between 30 and 150 bases, including each integer within the specified range. In another aspect, the non-naturally occurring DNA sequence comprises between 30 and 75 bases, including each integer within the specified range.

The present disclosure also provides diagnostic kits for identifying risk of developing adverse cellular senescence progression and raised p16 expression levels in response to a treatment or intervention. In certain embodiments, the diagnostic kit comprises reagents for measuring the level of one or more genes indicative of immunosenescence and cellular senescence. In certain embodiments, the diagnostic kit comprises reagents to measure p16, CD28, and CD244. In certain embodiments, the kit further includes reagents for isolating a sample in which one or more genes or gene products may be measured. In certain embodiments, the kit further includes reagents for genotyping a subject.

In some embodiments, the kits include quantitative RT-PCR reagents (RT-PCR kits). In certain embodiments, a kit that includes quantitative RT-PCR reagents includes the following: (a) primers used to amplify each of a combination of biomarkers (e.g., p16. CD28, and CD244) described herein; (b) buffers and enzymes including a reverse transcriptase; (c) one or more thermostable polymerases; and (d) Sybr® Green or a labelled probe, e.g., a TaqMan® probe. In another embodiment, the RT-PCR kits described herein also includes (a) a reference control RNA. In certain embodiments, kits include methods of detecting proteins, for example, and not limitation, antibodies designed to detect CD28 and CD244 protein expression.

In certain embodiments, RT-PCR kits comprise pre-selected primers specific for amplifying a particular cDNA corresponding to a portion or all of p16. The RT-PCR kits may also comprise enzymes suitable for reverse transcribing and/or amplifying nucleic acids (e.g., polymerases such as Taq), and deoxynucleotides and buffers needed for the reaction mixture for reverse transcription and amplification. The RT-PCR kits may also comprise probes specific for a particular cDNA corresponding to a portion or all of p16. The probes may or may not be labelled with a detectable label (e.g., a fluorescent label). Each component of the RT-PCR kit is generally in its own suitable container. Thus, these kits generally comprise distinct containers suitable for each individual reagent, enzyme, buffer, primer and probe. The kit may comprise reagents and materials so that a suitable housekeeping gene can be used to normalize the results, such as, for example, tyrosine 3-monooxygenase/tryptophan 5-monooxygenase activation protein, zeta polypeptide (YWHAZ) or β-actin. Further, the RT-PCR kits may comprise instructions for performing the assay and methods for interpreting and analyzing the data resulting from the performance of the assay.

The values from the assays described above, such as expression data, statistical analyses, composite score, and/or threshold score can be calculated and stored manually. Alternatively, the above-described steps can be completely or partially performed by a computer program product. In some embodiments, the methods of the present disclosure are computer-implemented methods. In some embodiments, at least one step of the described methods is performed using at least one processor. In certain embodiments, all of the steps of the described methods are performed using at least one processor. Further embodiments are directed to a system for carrying out the methods of the present disclosure. The system can include, without limitation, at least one processor and/or memory device.

Accordingly, aspects of the present disclosure may be implemented entirely in hardware, entirely in software (including firmware, resident software, micro-cods, etc.) or by combining software and hardware implementation that may all generally be referred to herein as a “circuit,” “module,” “component.” or “system.” Furthermore, aspects of the present disclosure may take the form of a computer program product embodied in one or more computer readable media having computer readable program code embodied thereon.

Any combination of one or more computer readable media may be utilized. The computer readable media may be a computer readable signal medium or a computer readable storage medium. A computer readable storage medium may be, for example, but not limited to, an electronic, magnetic, optical, electromagnetic, or semiconductor system, apparatus, or device, or any suitable combination of the foregoing. More specific examples (a non-exhaustive list) of the computer readable storage medium would include the following: a portable computer diskette, a hard disk, a random access memory (RAM), a read-only memory (ROM), an erasable programmable read-only memory (EPROM or Flash memory), an appropriate optical fiber with a repeater, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device. Program code embodied on a computer readable signal medium may be transmitted using any appropriate medium, including but not limited to wireless, wireline, optical fiber cable, RF, etc., or any suitable combination of the foregoing.

Computer program code for carrying out operations for aspects of the present disclosure may be written in any combination of one or more programming languages, including an object oriented programming language such as Java, Scala, Smalltalk, Eiffel JADE, Emerald, C++, C #, VB.NET, Python or the like, conventional procedural programming languages, such as the “C” programming language, Visual Basic, Fortran 2003, Perl, COBOL 2002, PHP, ABAP, dynamic programming languages such as Python, Ruby and Groovy, or other programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Interact using an Internet Service Provider) or in a cloud computing environment or offered as a service such as a Software as a Service (SaaS).

Aspects of the present disclosure may be described herein with reference to flowchart illustrations and/or block diagrams of methods, apparatuses (systems) and computer program products according to embodiments of the disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable instruction execution apparatus, create a mechanism for implementing the functions/acts specified m the flowchart and/or block diagram block or blocks.

These computer program instructions may also be stored in a computer readable medium that when executed can direct a computer, other programmable data processing apparatus, or other devices to function in a particular manner, such that the instructions when stored in the computer readable medium produce an article of manufacture including instructions which when executed, cause a computer to implement the function/act specified in the flowchart and/or block diagram block or blocks. The computer program instructions may also be loaded onto a computer, other programmable instruction execution apparatus, or other devices to cause a series of operational steps to be performed on the computer, other programmable apparatuses or other devices to produce a computer implemented process such that the instructions which execute on the computer or other programmable apparatus provide processes for implementing the functions/acts specified in the flowchart and/or block diagram block or blocks.

The following examples, which are included herein for illustration purposes only, are not intended to be limiting.

### EXAMPLES

### Example 1

Identification of a Subset of Patients where p16 Expression is Decreased or Increased by Caloric Restriction

Forty patients between 40 and 65 years of age were enrolled into a clinical trial. Additional inclusion criteria: BMI between 30-45.0 kg/m2 or BMI of 27-30, if waist circumference is ≥40 in in men or ≥35 in in women and fasting blood glucose 100-125 mg/dl or hemoglobin A1c 5.7%-6.4%, and sedentary for past 6 months (defined as less than 20 min, 2 days a week of resistance or aerobic exercise). Patients that met any of the following criteria were excluded: weight loss (±5%) in past 6 months; uncontrolled arrhythmias; cancer requiring treatment in past year, except non-melanoma skin cancers; regular smoker (>1 cigarette/day) or heavy drinker (>9 alcoholic drinks/wk) within past year; insulin dependent or uncontrolled diabetes (FBG>126 mg/dl) or (hemoglobin A1c 6.5% or greater); uncontrolled hypertension (BP>160/90 mmHg); elevated triglyceride (TG>400 mg/dl); clinically evident liver disease, kidney disease, edema or anemia, past or current ischemic heart disease, uncontrolled angina, heart failure, PAD, stroke, chronic respiratory disease, endocrine or metabolic disease, neurological or hematological disease; regular use growth/steroid hormones, including estrogen replacement, weight loss medications, diabetes medications including insulin and/or blood thinners and history of any type of bariatric or weight loss surgery or bilateral oophorectomy requiring long term hormone replacement therapy use. Participants underwent caloric restriction intervention for 18 weeks by either being guided to consume low carbohydrate/high protein meals or being given prepared meals.

Venous blood samples were collected from each patient into an EDTA tube during the patient's consultation visit prior to first intervention (v1) and at the end of the 18 weeks participation (v2). T cells were isolated from 5 ml of whole blood from each patient using standard negative selection methods including density gradient or magnetic bead kits, and stored frozen in a −80° C. freezer. Total RNA was isolated from T cells using ZR-96 quick-RNA™ kit from Zymo Research (cat. #R1053) using the manufacturer's protocol. RNA concentration was measured using a NanoDrop 2000 spectrophotometer. cDNA was prepared from 400 ng of total RNA using ImProm-II reverse transcriptase (cat #A3801; Promega) and 0.5 μg of random primers (cat #C1181; Promega) using the manufacturer's protocol. Resulting cDNA reactions were diluted 1:4 with distilled water. 4.5 μl of diluted cDNA was mixed with 5 μl of 2× iTaq Universal Probes Supermix (cat #1725135; Bio-Rad) and 0.5 μl 20× Assay primer/probe mix (p16 primers: Forward 5′-CCAACGCACCGAATAGTTACG-3′; Reverse 5′-GCGCTGCCCATCATCATG-3′; p16 probe: 5′ FAM-CCTGGATCGGCCTCCGAC-BHQ-1-3′; p14 primers: Forward 5′-CTGAGGAGCCAGCGTCTAG-3′; Reverse 5′-CCCATCATCATGACCTGGTCTTCTA-3′; p14 probe 5′ FAM—CAGCAGCCGCTTCC-BHQ-1-3′; CD28 Hs01007422_m1 (ThermoFisher Scientific); CD244 Hs00175569_m1 (ThermoFisher Scientific); CD4 Hs01058407_m1 (ThermoFisher Scientific); CD8 alpha Hs00233520_m1 (ThermoFisher Scientific); YWHAZ primers: Forward 5′-TGATGACAAGAAAGGGATTG-3′; Reverse 5′-CCCAGTCTGATAGGATGTGTT-3′; YWHAZ probe: 5′ FAM-TCGATCAGTCACAACAAGCATACCA-BHQ-1-3′. Real-time PCR reactions were performed using a CFX384 qPCR machine (Bio-Rad). A cycle threshold (Ct) of 37 was used as a cutoff point and any expression signal ≥37 was disregarded. Expression of each gene was normalized to expression of the housekeeping gene in the same patient by subtraction. The resulting values (log 2) were used for analysis.

As shown in Table 1, before intervention, as expected, biomarkers of cellular senescence p16 and p14 correlated with each other.

TABLE 1
Correlation of gene expression levels prior to intervention.

| | p14 v1 | P16 v1 | CD28 v1 | CD244 v1 | CD8/CD4 v1 |
|---|---|---|---|---|---|
| p14 v1 | 1 | 0.5304* | −0.0336 | −0.0849 | 0.3805* |
| P16 v1 | 0.5304* | 1 | −0.0455 | −0.0639 | 0.0498 |
| CD28 v1 | −0.0336 | −0.0455 | 1 | −0.3335** | −0.5437** |
| CD244 v1 | −0.0849 | −0.0639 | −0.3335** | 1 | 0.3901* |
| CD8/CD4 | 0.3805* | 0.0498 | −0.5437** | 0.3901* | 1 |
| v1 | | | | | |

*represents a positive correlation;
**represents an inverse correlation

CD28 and CD244, markers of different steps of the immunosenescence process, correlated as well, but inversely (lower CD28 correlated with higher CD244). Another measurement of immunosenescence, the ratio between expression of CD8 and CD4 positively correlated with CD244.

Gene expression levels of p16 or p14 (biomarkers of cellular senescence) did not correlate with CD28 or CD244, while p14 correlated with the CD8/CD4 ratio.

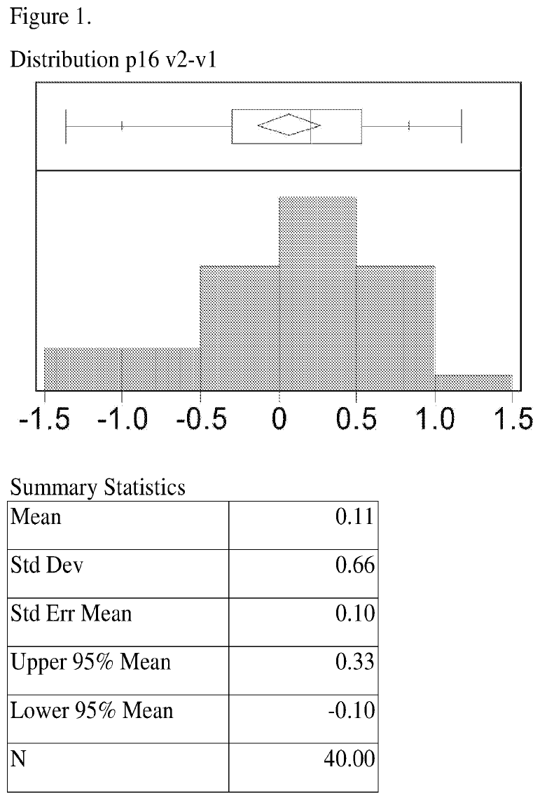

On average, p16 expression did not change with caloric restriction across the entire cohort (mean change 0.1, FIG. 1).

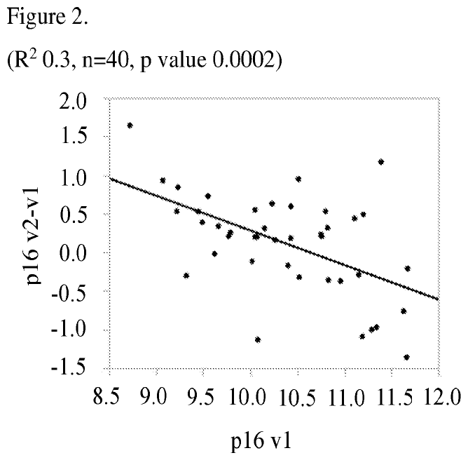

However, p16 change was highly dependent on the individual's p16 expression level prior to treatment (FIG. 2) and in patients with high p16 expression levels prior to treatment, caloric restriction caused a decrease in p16 expression levels (senolytic effect) and in patients with low p16 expression levels prior to treatment, caloric restriction increased p16 expression. Thus, in some cases, some individuals' p16 levels appear to benefit from caloric restriction, while others do not, and some individuals' p16 levels appear to increase in response to caloric restriction. Low p16 levels correlate with lower senescent load and a decelerated aging trajectory. And high p16 levels correlate with a higher senescent load and accelerated aging and morbidity (see e.g., U.S. Pat. No. 8,158,347). Thus, in certain embodiments, knowing before treatment whether a likely treatment will lower p16 or raise p16 can guide subject decision making. For example, and not limitation, subjects that are likely to benefit from caloric restriction can be enrolled for the treatment, while those that are unlikely to see any benefit in p16 levels, or increases in p16 levels can be excluded.

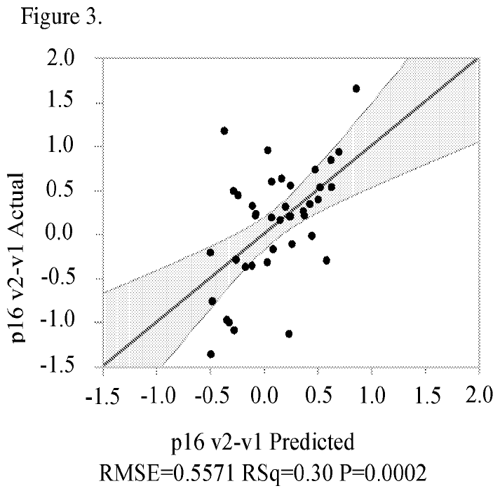

FIG. 3 demonstrates a model fit where p16 expression level prior to treatment is a variable and change in p16 is an outcome. This model predicts change in p16 with R square (Rsq) of 0.3 and also shows the Root Mean Square Error (RMSE) and P values (P).

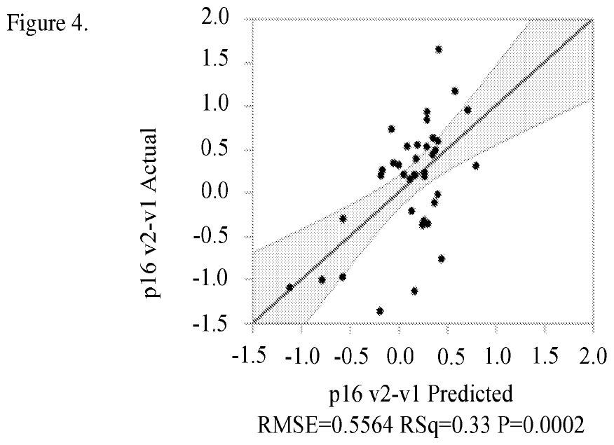

The ability of the immune system, as measured by immunosenescence, to predict change in p16 is shown in Table 2. Individually, none of the biomarkers are significant predictors of the p16 change. However, the interaction between CD28 and CD244 variables (designated as CD28*CD244) can predict change in p16 with statistical significance. FIG. 4 shows a model fit of CD28*CD244 to predict change in p16.

TABLE 2
The ability of biomarkers of immunosenescence
to predict change in p16 expression.

| Variable | p value |
|---|---|
| CD28v1*CD244 v1 | 0.00048 |
| CD8/CD4 v1 | 0.68008 |
| CD28 v1 | 0.81301 |
| CD244 v1 | 0.87438 |

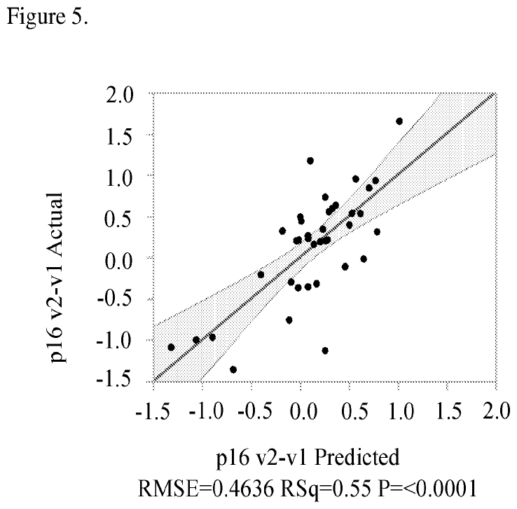

When combined, p16 expression levels prior to treatment and CD28*CD244 measured prior to treatment have an additive ability to predict change in p16 (FIG. 5, R square 0.55).

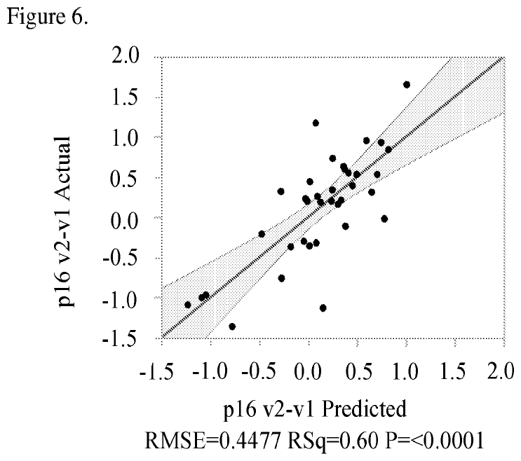

Substituting p16 expression levels prior to treatment with p16Age GAP measured prior to treatment, an age-appropriate estimation of p16, further increased the model fit (FIG. 6, R square 0.6).

Based on these data, we have developed a computational algorithm to predict the direction and magnitude of change in p16 expression by interventions such as caloric restriction or nutrient deprivation in an individual patient, as shown in the following algorithms.

### Algorithms:

determine level of CD28 before intervention,
determine level of CD244 before intervention
determine level of p16 before intervention
calculate p16Age GAP before intervention
Model 1: Algorithm for change in p16 using p16 levels before intervention:

a+(CD28−b)*((CD244−c)*d)−e*p16
Model 2: Algorithm for change in p16 using p16Age GAP before intervention:

f+(CD28−b)*((CD244−c)*d)−g*p16AgeGAP
a=the regression intercept for Model 1. In certain embodiments a is a number between 0.2 and 10. In certain embodiments, a is a number between 0 and 100.
b=an average expression of CD28 in a study population. In certain embodiments, b is a number between 15 and 20.
c=an average expression of CD244 in a study population. In certain embodiments, c is a number between 14 and 17.
d=a parameter estimate of a CD28*CD244 interaction variable. In certain embodiments, d is a number between 0.4 and 1.0. Note that this parameter estimate is calculated statistically to determine the relative contribution of the CD28*CD244 interaction relative to other variables in regression analysis to maximize the ability of the model to predict changes in p16. Thus, in certain embodiments, this value can be determined empirically based on a particular cohort. In certain other embodiments, this value can be calculated separately (such as, for example, and not limitation, a previous cohort) and plugged into the algorithm.
e—a parameter estimate of a p16 variable. In certain embodiments, e is a number between 0 and 0.6. Note that this parameter estimate is calculated statistically to determine the relative contribution of the p16 variable relative to other variables in regression analysis to maximize the ability of the model to predict changes in p16. Thus, in certain embodiments, this value can be determined empirically based on a particular cohort. In certain other embodiments, this value can be calculated separately (such as, for example, and not limitation, a previous cohort) and plugged into the algorithm.
f=the regression intercept for Model 2. In certain embodiments, f is a number between 0 and 1.
g—a parameter estimate of a p16Age GAP variable. In certain embodiments, g is a number between −0.5 and 0.5. Note that this parameter estimate is calculated statistically to determine the relative contribution of the p16Age GAP variable relative to other variables in regression analysis to maximize the ability of the model to predict changes in p16. Thus, in certain embodiments, this value can be determined empirically based on a particular cohort. In certain other embodiments, this value can be calculated separately (such as, for example, and not limitation, a previous cohort) and plugged into the algorithm.

The numbers can vary for a particular variable within a certain range depending on certain factors, (for example, and not limitation, the efficiency of the primers and probes used to detect particular markers; variations of marker expression within a particular cohort or study population; and other variables that can cause slight variation in signal strength and signal normalization). Two specific examples of such algorithms are shown below (one using p16 levels, the other using p16Age GAP).

$$Calculation using p 16 : 4.3381913309 + ( CD 28 Value - 17.179473684 ) * ( ( CD 244 Value - 15.153421053 ) * 0.6051367444 ) - 0.39 \; 673557 * p 16 Value = Predicted Change in p16 . Calculation using p 16 Age GAP : 0.5527014147 + ( CD 28 Value - 17.161111111 ) * ( ( CD 244 Value - 15.143055556 ) * 0.5796504986 ) - 0.012928514 * p 16 Age GAP Value = Predicted Change in p 16.$$

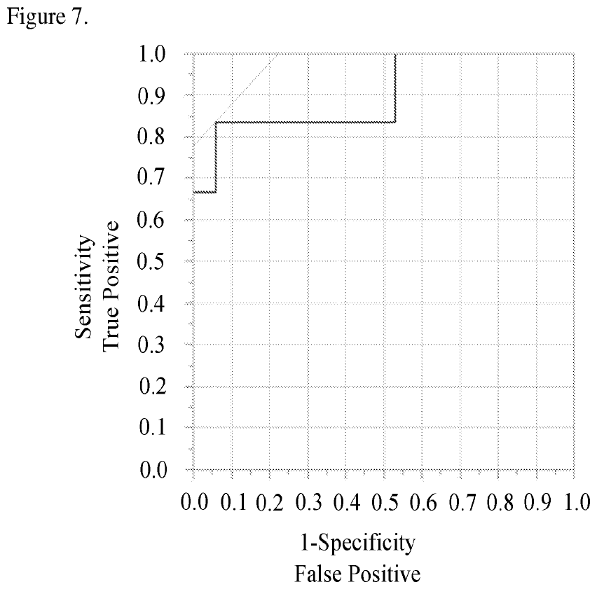

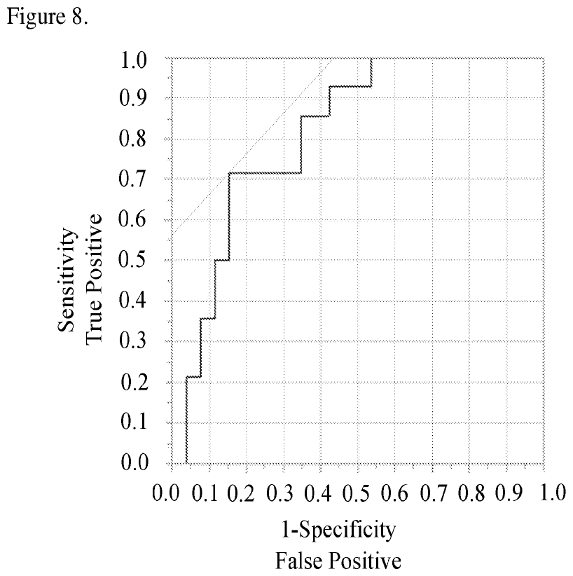

Using ROC analysis to predict individuals whose p16 may be increased or decreased by the intervention, we found that the algorithm combining pre-intervention expression levels of p16 and the interaction between CD28 and CD244 can predict individuals whose p16 levels after the intervention will decrease (FIG. 7, AUC=0.9) or increase (FIG. 8, AUC=0.8).

### Example 2

In this example, samples derived from ten patients were analyzed. Five patients underwent either autologous or allogeneic stem cell transplantation for hematologic malignancy. Patient samples were obtained from two cohorts: a study investigating symptom burden after transplantation, and a generic tissue procurement protocol. Patients undergoing concurrent radiation, chemotherapeutic, or investigational therapy other than transplant-related therapy were excluded. Samples were obtained at just before transplantation (pre), and at 6 months post-transplantation (post) (Wood, et al., EBioMedicine, 2016). The remaining five patients had early-stage breast cancer and were undergoing treatment that included cytotoxic chemotherapy. (Sanoff et al., 2014). Samples from patients with breast cancer were collected prior and post-chemotherapy treatment. Thus, both cohorts of patients received treatments known to induce senescence.

Sample preparation for RNA sequencing: CD3⁺ T-cells were isolated from up to 10-m1 of peripheral blood using anti-CD3 microbeads and an AutoMACS^{PRO} separator (Miltenyi Biotec, San Diego, CA). Total RNA was isolated using RNeasy Mini Kit (Qiagen) and rRNA was removed using the Ribo-Zero kit. RNA libraries were prepared by using the Illumina TruSeq RNA Sample Preparation Kit v2 and then sequenced by Illumina HiSeq2000. Reads were subjected to quality control as previously described (Cancer Genome Atlas Research, 2012). RNA reads were aligned to human hg19 genome assembly using Mapsplice (Wang et al., 2010). Gene expression was estimated using RSEM (RNA-Seq by Expectation Maximization) (Li and Dewey, 2011). Genes differentially expressed due to treatment were identified by DESeq2 (Love et al., 2014) using a bivariate model to adjust for subject specific effects.

P16 gene expression was upregulated in the post samples of patients undergoing chemotherapy or stem cell transplantation, see, Wood, et al., EBioMedicine, (2016).

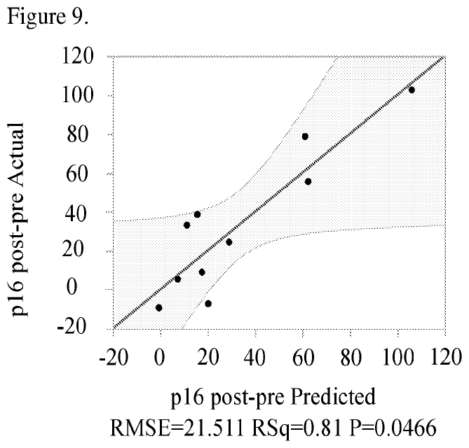

Similar to Example 1, a combination of expression of p16, CD244, and CD28 was able to predict treatment-induced change in p16 (FIG. 9, R square 0.8).

A computational algorithm to predict the direction and magnitude of change in p16 expression by interventions such as chemotherapy and stem cell transplant in an individual patient was prepared based on the statistical analysis in Example 1, Model 1 (a+(CD28−b)*((CD244-c)*d)-c*p16). Regression intercept, parameter estimate for each variable, and average expression levels of CD28 an CD244 in the cohort were used in the Model to predict change in p16. Because the method of measurement of gene expression is different (RNA sequencing vs qRT-PCR), the absolute value of gene expression is different (target counts vs cycle threshold measurement). The example algorithm calculated according to Example 2 is shown below.

$$Calculation using p 16 : 46.74691295 + ( CD 244 Value - 1 , 114.63441 ) * ( ( CD 28 Value - 7 , 21 \; 0.282 \; 46 ) * - 3.266742 {e}^{- 6} ) - ( 0.684413477 * p 16 Value ) = Predicted Change in p 16.$$

Therefore, regardless of the method used to measure gene expression of p16, CD28, and CD244, the methods and algorithms described herein can be used to model the direction and magnitude of changes in p16 expression resulting from various interventions. And those models can be used to predict outcomes for patients undergoing those interventions.

What is claimed is:

1. A method of treating a subject to reduce the overall cellular senescent load of the subject, comprising:
a) requesting a result of a clinical test, wherein the clinical test comprises obtaining a blood sample from the subject;
b) detecting a level of gene expression of p16 in the sample for the subject and generating a value for p16;
c) detecting a level of gene expression of CD28 in the sample for the subject and generating a value for CD28;
d) detecting a level of gene expression of CD244 in the sample for the subject and generating a value for CD244;
e) generating a composite score based on the value for p16, the value for CD28, and the value for CD244; wherein the composite score represents a prognostic value of the p16 levels in the subject in response to the intervention; and
f) treating the subject with an intervention to reduce the overall cellular senescent load of the subject if the composite score indicates that the p16 levels of the subject may decrease as a result of the intervention.

2. The method of claim 1, further comprising generating a value for the interaction between CD28 and CD244, and combining it with the value for p16 to estimate the magnitude of change in p16 levels in the subject in response to the intervention.

3. The method of claim 2 wherein the generating a value for the interaction between CD28 and CD244 comprises:
a) determining a population mean value of CD244 in a study population and subtracting the population mean value of CD244 in the study population from the CD244 expression level of the subject to generate a value representing the difference in CD244 expression between the subject and the population mean;
b) determining a population mean value of CD28 in the study population and subtracting the population mean value of CD28 in the study population from the CD28 expression level of the subject to generate a value representing the difference in CD28 expression between the subject and the population mean;
c) multiplying the value representing the difference in CD244 expression between subject and the population mean, the value representing the difference in CD28 expression between subject and the population mean, and a parameter estimate of a CD28*CD244 interaction variable to generate a value for the interaction between CD28 and CD244.

4. The method of claim 1, further comprising isolating peripheral blood T lymphocytes from the blood sample of step (a).

5. The method of claim 1, wherein the generating a value for p16 comprises calculating a p16Age GAP Value for the subject.

6. The method of claim 5, wherein the generating a p16Age GAP Value comprises:
a) generating a p16 value for the subject from the level of gene expression of p16 in the sample;
b) converting the p16 value for the subject into a p16Age Value for the subject; and
c) generating a p16Age GAP Value for the subject by subtracting the chronological age of the subject from the p16Age Value of the subject.

7. The method of claim 1, wherein the treating the subject with an intervention to reduce the overall cellular senescent load of the subject comprises administering one or more compounds that induce a senolytic effect.

8. The method of claim 7, wherein the one or more compounds comprises at least one of rapamycin, fisetin, metformin, canagliflozin, dapafliflozin, empagliflozin, ertugliflozin, roxadustat, molidustat, vadavustat, daprodustat, and dsatinib in combination with quercetin.

9. The method of claim 1 wherein the treating the subject with an intervention to reduce the overall cellular senescent load of the subject comprises one or more lifestyle interventions.

10. The method of claim 9, wherein the one or more lifestyle interventions comprises one or more of fasting, caloric restriction, dietary supplements, use of probiotics, an exercise regimen, and sleep monitoring.

11. A method of risk assessment for a subject considering the use of one or more interventions to lower the senescent load in the subject comprising:
a) requesting a result of a clinical test, wherein the clinical test comprises obtaining a blood sample from the subject;
b) detecting a level of gene expression of p16 in the sample for the subject and generating a value for p16;
c) generating a value for CD28 for the subject;
d) generating a value for CD244 for the subject;
e) generating a composite score based on the value for p16, the value for CD28, and the value for CD244; wherein the composite score represents a prognostic value of the p16 levels in the subject in response to the intervention; and
f) comparing the composite score to a threshold value and determining that the subject should receive the intervention if the composite score falls on one side of the threshold value and determining that the subject should not receive the intervention if the composite score falls on the other side of the threshold value.

12. The method of claim 11, further comprising generating a value for the interaction between CD28 and CD244, and combining it with the value for p16 to estimate the magnitude of change in p16 levels in the subject in response to the intervention.

13. The method of claim 12, wherein the generating a value for the interaction between CD28 and CD244 comprises:
a) determining a population mean value of CD244 in a study population and subtracting the population mean value of CD244 in the study population from the CD244 expression level of the subject to generate a value representing the difference in CD244 expression between the subject and the population mean;
b) determining a population mean value of CD28 in the study population and subtracting the population mean value of CD28 in the study population from the CD28 expression level of the subject to generate a value representing the difference in CD28 expression between the subject and the population mean;
c) multiplying the value representing the difference in CD244 expression between subject and the population mean, the value representing the difference in CD28 expression between subject and the population mean, and a parameter estimate of a CD28*CD244 interaction variable to generate a value for the interaction between CD28 and CD244.

14. The method of claim 11, further comprising isolating peripheral blood T lymphocytes from the blood sample of step (a).

15. The method of claim 11, wherein the generating a score for p16 comprises calculating a p16Age GAP Value for the subject.

16. The method of claim 15, wherein the generating a p16Age GAP Value comprises:
a) generating a p16 value for the subject from the level of gene expression of p16 in the sample;
b) converting the p16 value for the subject into a p16Age Value for the patient; and
c) generating a p16Age GAP Value for the subject by subtracting the chronological age of the subject from the p16Age Value of the subject.

17. The method of claim 11, wherein the one or more interventions comprises administering one or compounds that induce a senolytic effect.

18. The method of claim 17, wherein the one or more compounds comprises at least one of rapamycin, fisetin, metformin, canagliflozin, dapafliflozin, empagliflozin, ertugliflozin, roxadustat, molidustat, vadavustat, daprodustat, and dsatinib in combination with quercetin.

19. The method of claim 11 wherein the one or more interventions comprises one or more lifestyle interventions.

20. The method of claim 19, wherein the one or more lifestyle interventions comprises one or more of fasting, caloric restriction, dietary supplements, use of probiotics, an exercise regimen, and sleep monitoring.

21. The method of claim 11, wherein the threshold value is set such that the subject receives the intervention if p16 levels are projected to decrease and does not receive the intervention if p16 levels are projected to increase or remain the same.
